(19) United States
(12) Patent Application Publication
(10) Pub. No.: US 2024/0029846 A1
(43) Pub. Date: Jan. 25, 2024

(54) METHOD AND SYSTEM FOR DISCOVERING NEW DRUG INDICATION BY FUSING PATIENT PORTRAIT INFORMATION

(71) Applicant: ZHEJIANG LAB, Hangzhou (CN)

(72) Inventors: Yu WANG, Hangzhou (CN); Jingsong LI, Hangzhou (CN); Yu TIAN, Hangzhou (CN); Tianshu ZHOU, Hangzhou (CN)

(21) Appl. No.: 18/362,950

(22) Filed: Jul. 31, 2023

Related U.S. Application Data
(63) Continuation of application No. PCT/CN2021/113136, filed on Aug. 18, 2021.

(30) Foreign Application Priority Data
May 31, 2021 (CN) 202110599266.2

(51) Int. Cl. G16H 10/60; G16C 20/30

(52) U.S. Cl. CPC G16H 10/60; G16C 20/30

(57) ABSTRACT

Disclosed is a method and a system for discovering new drug indications by fusing patient portrait information. According to the present disclosure, real-world patient medication and patient diagnostic data are introduced into a data-driven drug relocation solution, an actual use effect of drugs in a broader population is added into a new drug-disease relationship prediction model. According to the present disclosure, a patient portrait is constructed as a feature expression of patient information, and is used to construct a patient-patient network as a medium between drug and disease networks, and a heterogeneous network system corresponding to actual clinical processes is constructed. Prediction results in the present disclosure are more closely related to a clinical practice, and a probability of success in subsequent validation of old drugs for new usage and new clinical trials is greater.

### CROSS-REFERENCE TO RELATED APPLICATIONS

The present application is a continuation of International Application No. PCT/CN2021/113136, filed on Aug. 18, 2021, which claims priority to Chinese Application No. 202110599266.2, filed on May 31, 2021, the contents of both of which are incorporated herein by reference in their entireties.

### TECHNICAL FIELD

The present disclosure belongs to the technical field of medical information, and in particular, to a method and a system for discovering new drug indications by fusing patient portrait information.

### BACKGROUND

In recent years, many drug developers have tried their best to explore new uses or new ways of using existing drugs. The process of finding new uses for existing drugs beyond the original medical indications is called drug repositioning. Because the pharmacokinetics and toxicological characteristics of drugs already on the market have passed a lot of research and verification, drug repositioning research can greatly save drug development cost, reduce development cycle, and mitigate the risk of drug development failure. Since the concept of drug repositioning was first introduced, its scope and application have expanded significantly. The potential to repurpose existing drugs for new therapeutic indications has led to growing interest and investment.

In addition to accidental discovery, data-driven is the main way of systematic drug repositioning research, which is mainly based on the similarity theory, that is, drugs with similar structures/targets/action pathways may treat the same diseases. At present, the research mainly uses the preclinical characteristics of single or integrated drugs or diseases, and finds the relationship between new drugs and diseases through similarity integration. Gottlieb and other associates integrated drug molecular structure, drug molecular activity and disease semantic information to construct a drug-disease network; an invention patent with a publication number of CN107506591B “Drug repositioning method based on multi-information fusion and random walk model” discloses a drug repositioning method based on multi-information fusion and random walk model. By integrating the existing disease data, drug data, target data, disease-drug association data, disease-gene association data and drug-target association data, a disease-target-drug heterogeneous network is constructed, and the basic random walk model is extended to the constructed heterogeneous network, and the global network information is effectively used to recommend candidate therapeutic drugs for diseases.

The above research ideas make use of the massive data accumulated in previous preclinical drug trials as much as possible through computer technology, and tap new values from them. Post-marketing diagnostic and treatment data were rarely used, but this portion of the real-world data accurately reflects the actual clinical diagnosis and treatment effect of the drug.

The existing data on drug properties, disease characteristics and drugs-diseases relationship mostly obtained from pre-clinical studies and clinical trials before drug marketing, and pre-clinical trials are mostly controlled in a strict experimental environment. However, due to the strict inclusion and exclusion criteria in traditional clinical trials, the experimental population may not be fully representative of the target population, and the standard interventions used may not be fully consistent with the clinical practice. Limited sample size and short follow-up time lead to insufficient evaluation of adverse events. In addition, traditional clinical trials are difficult to conduct in some diseases and fields, therefore the existing methods can only reflect the response of drugs in strictly controlled experimental environment, and cannot fully reflect the use effect of drugs in real clinical practice. It is very limited to find new drug indications only by using this part of data. At the same time, the existing methods are all based on the known relationships between drugs, diseases and targets, but in the real world, the pathways and mechanisms of drug action in the human body have not been thoroughly studied. Some studies have shown that the results of predicting the relationship between drugs and diseases by existing methods are usually more optimistic than the actual situation.

### SUMMARY

In view of the shortcomings of the prior art, the present disclosure introduces real-world patient data into the existing data-driven new drug indication discovery method and system, and constructs the association between drugs and diseases in real-world clinical activities by constructing patient portraits. Based on the assumption that similar patients may suffer from similar diseases and can be treated with similar drugs, a drug composite similarity network, a patient portrait similarity network, a disease phenotype similarity network and a drug-patient-disease heterogeneous network are constructed in combination with the existing published data in the field of drug repositioning. Then new drug indications, that is, new real-world evidence, are discovered.

The purpose of the present disclosure is achieved through the following technical solutions.

In a first aspect, the present disclosure discloses a method for discovering new drug indications by fusing patient portrait information, and the method includes the following steps:

(1) Performing data acquisition and association: obtaining public data of drugs and diseases, obtaining real-world patient data from electronic health record data, and associating the drugs and the diseases in the real-world patient data with corresponding drugs and diseases in the public data.

(2) Generating patient portraits: generating corresponding patient tags by cleaning and converting the electronic health record data obtained in step (1). Further, in step (2), a same patient can have a plurality of patient portraits after a plurality of visits.

(3) Calculating a drug composite similarity, a disease phenotype similarity and a patient portrait similarity, and constructing a drug-drug network C, a disease-disease network D and a patient-patient network P according to the three similarities.

(4) Constructing a drug-patient relationship network CP according to medication data of a current visit after each patient portrait is generated, constructing a patient-disease relationship network PD according to diagnostic data of a current visit after each patient portrait is generated, and constructing a drug-disease relationship network CD according to a known association between the drugs and the diseases.

(5) Constructing a drug-patient-disease heterogeneous network by the networks C, D, P, CP, PD and CD,

Further, in step (5), an adjacency matrix A of the heterogeneous network is:

$$A = [ \begin{matrix}{A}_{c} & {A}_{CP} & {A}_{CD} \\ {A}_{CP}^{T} & {A}_{P} & {A}_{PD} \\ {A}_{CD}^{T} & {A}_{PD}^{T} & {A}_{D}\end{matrix} ]$$

where A_{c}, A_{P} and A_{D} represent the adjacency matrices of the networks C, P and D respectively, A_{CP}, A_{PD} and A_{CD} represent the adjacency matrices of the networks CP, PD and CD respectively, and T represents transposition.

(6) Predicting a relationship between the drugs and the diseases based on a bidirectional random walk method, that is, taking a certain drug node as a seed of random walk, and predicting a probability R of reaching a certain disease node when the random walk reaches a steady state, and calculating random walk lengths of a drug node and a patient node in the forward link, and random walk lengths of a disease node and a patient node in the reverse link, respectively, based on a topological structure of the heterogeneous network.

Said taking a certain drug node as a seed of random walk, and predicting a probability R of reaching a certain disease node when the random walk reaches a steady state comprises the following sub-steps:

constructing an initial vector R⁽⁰⁾=A_{CD} at a random walk starting time t=0, and performing normalization on A_{CD};

assuming that two random walk links are performed:

a) a forward link: the seed starts from a certain node in the network C and walks through the network P to the network D, and after walking for a time t, a probability of the walk seed staying at each node is calculated as follows:

R_{F-CP}^{(t)}=(1−λ_{CP})A_{CP}A_{CP}

R_{F-PD}^{(t)}=(1−λ_{PD})A_{P}R_{F-PD}^{(t-1)}+λ_{PD}A_{PD}

R_{F}^{(t)}=αR_{F-CP}^{(t)}R_{F-PD}^{(t)}+(1−α)A_{CD}

where a subscript F represents the forward link, λ_{CP} represents a probability of the seed transferring from the network C to the network P, λ_{PD} represents a probability of the seed transferring from the network P to the network D, R_{F-CP}^{(t)} and R_{F-CP}^{(t-1)} are probabilities that the random walk seed from the network C stays in the network P at times t and t−1, R_{F-PD}^{(t)} and R_{F-PD}^{(t-1)} are probabilities that the random walk seed from the network P stays in the network D at the times t and t−1, and a is a weight factor;

b) a reverse link: the seed starts from a certain node in the network D and walks through the network P to the network C, and after walking for a time t, a probability of the walk seed staying at each node is calculated as follows:

R_{B-DP}^{(t)}=(1−λ_{DP})A_{D}R_{B-DP}^{(t-1)}+λ_{DP}A_{PD}^{T}

R_{B-PC}^{(t)}=(1−λ_{PC})A_{P}R_{B-PC}^{(t-1)}+λ_{PC}A_{CP}^{T}

R_{B}^{(t)}=α(R_{B-DP}^{(t)}R_{B-PC}^{(t)})^{T}+(1−α)A_{CD}

where a subscript B represents the reverse link, λ_{DP} represents a probability of the seed transferring from the network D to the network P, λ_{PC} represents a probability of the seed transferring from the network P to the network C, R_{B-DP}^{(t)} and R_{B-DP}^{(t-1)} are probabilities that the random walk seed from the network D stays in the network P at times t and t−1, and R_{B-PC}^{(t)} and R_{B-PC}^{(t-1)} are probabilities that the random walk seed from the network P stays in the network C at the times t and t−1.

In a random walk iterative process, when a certain node satisfies that the random walk length of the certain node is smaller than or equal to t, the random seed starting from the node will no longer walk;

$$R = \frac{( {R}_{F} + {R}_{B} )}{2}$$

obtained after the end of the random walk is a probability of a drug treating corresponding diseases, and if the two have no known association therebetween, the drug is taken as a discovery result of new drug indications.

Further, in step (1), the information obtained from the electronic health record data includes: \textcircled{1} demographic information: age, gender and ethnicity; \textcircled{2} basic medical information: allergy history, family history and a blood type; \textcircled{3} diagnosis and treatment information: historical diagnostic records, abnormal laboratory results and historical medication records; and \textcircled{4} medical result information: diagnosis and medication records generated by a current visit.

Further, in step (2), the gender, ethnicity, allergen, blood type and abnormal test results of a patient use custom codes, and a coding form is not limited; historical diagnosis and family medical history use ICD-10 codes; and historical medication information uses drug codes in a DrugBank data set.

Further, in step (3), the drug composite similarity includes a drug structure similarity, a target similarity, a pathway similarity and an adverse reaction similarity; Using drug 2D molecular fingerprint data, the structural similarity of drugs can be calculated by computing the Tanimoto coefficient; and the target similarity, the pathway similarity and the adverse reaction similarity are all calculated through a Jaccard coefficient.

Further, in step (3), the calculation of the drug composite similarity specifically is as follows:

the drug composite similarity is calculated by using a non-linear heterogeneous network fusion mode according to 4 dimensions of the drug composite similarity, and a similarity network of each dimension is expressed as G=(V, E), where V is a node, corresponding to the drugs in the 4 similarity networks, and E is an edge, represented by the similarity among the drugs; and for the 4 similarity networks, an overall normalized weight matrix K is defined:

$$K ( i , j ) = \{ \begin{matrix}\frac{sim ( i , j )}{2 {\sum }_{k \neq i} sim ( i , k )} , j \neq i \\ \frac{1}{2} , j = i\end{matrix}$$

where sim (i, j) is a similarity between a drug i and a drug j in a certain dimension;

meanwhile, a local weight matrix S is defined:

$$S ( i , j ) = \{ \begin{matrix}\frac{sim ( i , j )}{{\sum }_{k ∈ {N}_{i}} sim ( i , k )} , j ∈ {N}_{i} \\ 0 , other\end{matrix}$$

where Nᵢ is a neighbor node of a node i calculated through a KNN algorithm, and a similarity among non-neighbor nodes is set as 0; and

for the similarity network of each dimension, the calculated matrixes K and S are taken as an initial state of the heterogeneous network fusion, and an iterative update formula for the heterogeneous network fusion is:

$$K ( v ) = {S}^{( v )} \times ( \frac{{\sum }_{k \neq v} {K}^{( v )}}{m - 1} ) \times {( {S}^{( v )} )}^{T} , v = 1 , 2 , … m , m = 4$$

after a plurality of iterations, K^{(v)} tends to be stable and consistent, and a final drug composite similarity is obtained.

Further, in step (3), the disease phenotype similarity is calculated using a hierarchical coding structure of ICD-10, and the disease phenotype similarity between diseases i and j is calculated using the following formula:

$$sim ( i , j ) = \{ \begin{matrix}1 - {‖ Number ( i ) - Number ( j ) ‖}^{2} & Initial letters of ICD - 10 codes for i and j are the same \\ 0 & other\end{matrix}$$

where Number(i) and Number(j) respectively represent numbers after removing first letters from ICD-10 codes of the diseases i and j.

Further, in step (3), the patient portrait similarity is calculated by weighted averaging of an age similarity, a gender similarity, an ethnic similarity, an allergen similarity, a family medical history similarity, a blood type similarity, a historical diagnostic similarity, a historical medication similarity and an abnormal testing result similarity of patients; the age similarity is calculated using a Euclidean distance; the gender similarity and the ethnicity similarity are calculated using a binary approach where a value of 1 indicates similarity (when the gender or ethnicity is the same) and a value of 0 indicates dissimilarity; and information of other dimensions are calculated by using a Jaccard distance through coding.

Further, in step (3), in the process of constructing the patient-patient network P, when the patient portrait similarity between two nodes is smaller than a threshold value ε, then a value of an edge between the two nodes is set as 0, and ε is set to be a quantile of the complete patient portrait similarity.

Further, in step (6), assuming that the drug-patient-disease heterogeneous network totally contains n drugs, x patients and m types of disease information, random walk lengths L_{CP}(cᵢ) and L_{PD}(pᵢ) of a drug node cᵢ and a patient node pᵢ in the forward link, and random walk lengths L_{DP}(dᵢ) and L_{PC}(pᵢ) of a disease node dᵢ and a patient node pᵢ in the reverse link are calculated as follows:

L_{CP}(cᵢ)=Σⱼ₌₁^{x}J(cᵢ,pⱼ)

L_{PD}(pᵢ)=Σⱼ₌₁^{m}J(pᵢ,dⱼ)

L_{DP}(dᵢ)=Σⱼ₌₁^{x}J(dᵢ,pⱼ)

L_{PC}(pᵢ)=Σⱼ₌₁ⁿJ(pᵢ,cⱼ)

where J represents a topological structure similarity of two nodes; and for L_{CP}(cᵢ), a calculation formula of J(cᵢ, pᵢ) is as follows:

$$J ( {c}_{i} , {p}_{j} ) = \frac{\vert {N}^{C} ( {c}_{i} ) ∩ ( {p}_{j} ) \vert}{\vert {N}^{C} ( {c}_{i} ) ∪ ( {p}_{j} ) \vert}$$

$$( {p}_{j} ) = {∪}_{s ∈ {N}^{P} ( {p}_{j} )} {N}^{C} ( s )$$

where N^{C}(cᵢ) represents a neighbor node of the node cᵢ in the drug-drug network C, and [symbol] (pⱼ) represents a neighbor node of all neighbor nodes in the drug-drug network C of a node pⱼ in the patient-patient network P.

In another aspect, the present disclosure provides a drug repositioning system that integrates patient profile information, comprising: a data acquisition module configured to acquire and associate public data of drugs and diseases and real-world patient data; a data preprocessing module configured to clean and convert data, and perform association mapping on the public data and the real-world patient data; a new drug indication discovery module configured to search for new drug indications in a drug-patient-disease global relationship; and a prediction result display module configured to present prediction result data. The new drug indication discovery module uses the method for discovering new drug indications to construct a drug-patient-disease heterogeneous network, and then performs drug-disease relationship prediction based on a bidirectional random walk method.

The present disclosure has the advantages that in the past data-driven drug repositioning research, only public data sets were usually used, and most of these data are from preclinical experiments or clinical experimental results, and there may be conflicts and contradictions between different data sets, so utilizing such data for drug repositioning studies has limitations. According to the present disclosure, real-world patient medication and patient diagnosis data are introduced into a data-driven drug repositioning solution, and the actual use effect of drugs in a wider population is added into a new drug-disease relationship prediction model; According to the present disclosure, the patient portrait is constructed as the characteristic expression of the patient information, and the patient-patient network is constructed as the medium between the drug and the disease network, so as to construct a heterogeneous network system which conforms to the actual clinical process; The predicted results will be closer to the clinic, and it is more likely to be successful in the subsequent verification of new use of old drugs and new clinical trials.

### BRIEF DESCRIPTION OF DRAWINGS

FIG. 1 is a flowchart of a method for discovering new drug indications by fusing patient portrait information provided by an embodiment of the present disclosure;

FIG. 2 is a schematic diagram of similarity calculation provided by an embodiment of the present disclosure;

FIG. 3 is a schematic diagram of the discovery process of new drug indications provided by the embodiment of the present disclosure; and

FIG. 4 is a structural block diagram of a system for discovering new drug indications by fusing patient portrait information provided by an embodiment of the present disclosure.

### DESCRIPTION OF EMBODIMENTS

In order to make the above objects, features and advantages of the present disclosure more obvious and easy to understand, the specific embodiments of the present disclosure will be described in detail with reference to the accompanying drawings.

In the following description, many specific details are set forth in order to fully understand the present disclosure, but the present disclosure can also be implemented in other ways different from those described here, and those skilled in the art can make similar promotion without violating the connotation of the present disclosure, so the present disclosure is not limited by the specific embodiments disclosed below.

According to the present disclosure, real-world patient medication and patient diagnosis data are introduced into a data-driven drug repositioning solution, and the actual use effects of drugs in a wider population are added into a new drug-disease relationship prediction model. In the present disclosure, real-world patient data refers to various data related to the patient's health status and/or diagnosis and treatment and health care collected daily; real-world evidence refers to clinical evidence about drug use and potential benefits-risks obtained through proper and sufficient analysis of applicable real-world data, including evidence obtained through retrospective or prospective observational studies or intervention studies such as clinical trials.

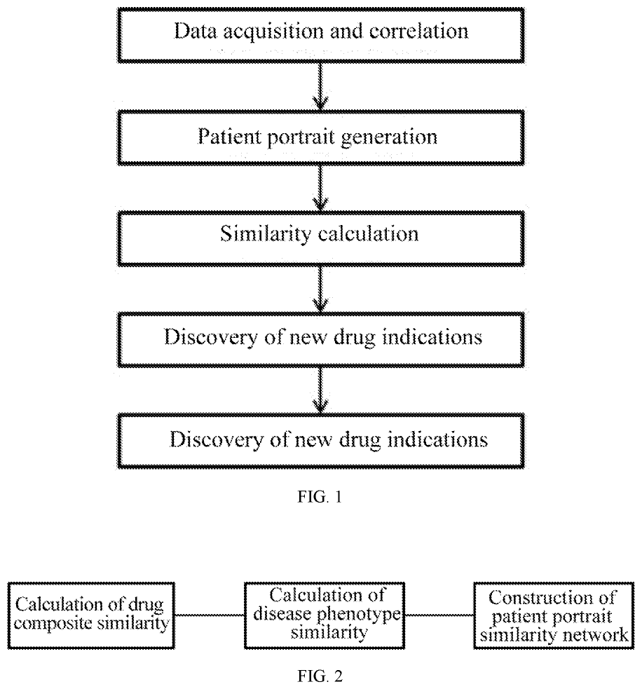

As shown in FIG. 1, a method for discovering new drug indications by fusing patient portrait information provided by an embodiment of the present disclosure includes the following steps:

Step 1: Data Acquisition and Correlation

The chemical structure, target and pathway information of drugs are obtained through the public data set DrugBank; information of drug indications and adverse drug reactions is obtained from a SIDER data set; the international disease classification standard ICD-10 is obtained. Real-world patient data are obtained from electronic health record data, and the time point of each visit (outpatient/inpatient) is taken as a cross section. The obtained information includes: \textcircled{1} demographic information: age, gender and nationality; \textcircled{2} basic medical information: allergy history, family history and blood type; \textcircled{3} diagnosis and treatment information: historical diagnosis records, abnormal test results and historical medication records; \textcircled{4} medical result information: the diagnosis and medication records produced in this visit. The drugs and diseases in the real-world patient data are correlated with the corresponding drugs and diseases in the public data set.

Step 2: Patient Portrait Generation

The generation of patient portrait is to generate a series of “tags” for patients, and the patient tags involved in the present disclosure include: age, gender, nationality; allergen, family history and blood type; historical diagnosis, historical medication, abnormal test results. The electronic health record data extracted in step 1 is cleaned and converted to generate the corresponding patient tag. The following is an example of a patient portrait:

PID (patient 1)

Age: 59

Sex: 1 (male)

Ethnic group: 1 (Han nationality)

Allergen: ALG01 (penicillin)

Family history: B18.1 (chronic hepatitis B)|C17.0 (duodenal malignant tumor)

Blood type: 01 (Rh positive type A)

Historical diagnosis: E74.801 (renal diabetes)|I10 (hypertension)

Historical medication: DB00381 (amlodipine)|DB00177 (valsartan)

Abnormal test results: GHB (glycosylated hemoglobin)|SCR (creatinine)|Alb (albumin)

Among them, the patient identifier (PID) is the unique identifier of patient identity; the codes used for gender, nationality, allergen, blood type and abnormal test results are self-designed codes, and the coding form is not limited; ICD-10 codes are used for historical diagnosis and family history; the drug codes in DrugBank data set are used for the historical drug use information; in the above example, the contents in brackets are the corresponding names of codes. In the embodiment of the present disclosure, the same patient has multiple pieces of patient portrait information for multiple visits.

Step 3: similarity calculation, as shown in FIG. 2, includes the following steps.

3.1 Drug Composite Similarity Calculation

A drug composite similarity network consists of drug structure similarity, target similarity, pathway similarity and adverse reaction similarity. Using drug 2D molecular fingerprint data, the structural similarity of drugs can be calculated by computing the Tanimoto coefficient. The chemical structural similarity sim_{chem} (i, j) between drugs i and j and is as follows:

$${sim}_{chem} ( i , j ) = \frac{c}{a + b - c}$$

where a and b are the number of ‘1’ in the molecular fingerprints of drugs i and j respectively, and c is the number of ‘1’ in the same position in the molecular fingerprints of drugs i and j. The target similarity, pathway similarity and adverse reaction similarity are all calculated by a Jaccard coefficient. Taking the target similarity as an example, the target similarity sim_{target} (i, j) of drugs i and j is:

$${sim}_{target} ( i , j ) = \frac{\vert A ⋂ B \vert}{\vert A ⋃ B \vert}$$

where, A and B are the target sets of drugs i and j respectively.

According to the above method, a similarity network of four dimensions is constructed, and a nonlinear heterogeneous network fusion mode is used to complete the calculation of the drug composite similarity. The similarity network of each dimension can be expressed as G=(V, E), where V is the node of the network, corresponding to the drugs in the four similarity networks, and E is the edge of the network, represented by the similarity among drugs. For the four similarity networks, an overall normalized weight matrix may be defined:

$$K ( i , j ) = \{ \begin{matrix}\frac{sim ( i , j )}{2 {\sum }_{k \neq i} sim ( i , k )} , j \neq i \\ \frac{1}{2} , j = i\end{matrix}$$

where sim (i, j) is a similarity between drugs i and j in a certain dimension.

Meanwhile, a local weight matrix S may be defined:

$$S ( i , j ) = \{ \begin{matrix}\frac{s i m ( i , j )}{{\sum }_{k ∈ {N}_{i}} s i m ( i , k )} , j ∈ {N}_{i} \\ 0 , other\end{matrix}$$

where Nᵢ is a neighbor node of a node i calculated through a KNN algorithm, and a similarity among non-neighbor nodes is set as 0 through the calculation of S.

For the similarity network of each dimension, the calculated matrixes K and S are taken as an initial state of the heterogeneous network fusion, and an iterative update formula for the heterogeneous network fusion is:

$${K}^{( v )} = {S}^{( v )} \times ( \frac{{\sum }_{k \neq v} {K}^{( v )}}{m - 1} ) \times {( {S}^{( v )} )}^{T} , v = 1 , 2 , … m , m = 4$$

after iterations of a time t, K^{(v)} tends to be stable and consistent, and a final drug composite similarity is obtained.

3.2 Disease Phenotype Similarity Calculation

The disease phenotype similarity is calculated by using the hierarchical coding structure of ICD-10; ICD-10 codes consist of four-digit levels (one letter and three digits), and the first three codes are separated from the last by decimal points, for example “A15.0”, in which the first three codes “A15” represent respiratory tuberculosis and “A15.0” represent tuberculosis; in “B15.0”, the first three codes “B15” represent viral hepatitis, while “B15.0” represents hepatitis A with hepatic coma. In ICD-10 coding system, when the initials are different, it can be considered that diseases belong to different categories, with great differences; when the initials are the same, the last three codes can be used as the basis for calculating the distance between diseases. The similarity between diseases i and j is defined as follows:

$$sim ( i , j ) = \{ \begin{matrix}1 - {‖ Number ( i ) - Number ( j ) ‖}^{2} & \begin{matrix}Initial letters of ICD - 10 codes \\ for i and j are the same\end{matrix} \\ 0 & other\end{matrix}$$

where Number(i) and Number(j) respectively represent numbers after removing first letters from ICD-10 codes of the diseases i and j (1 decimal place is reserved); when the first letters are the same, the similarity between diseases i and j is recorded as 1 minus the Euclidean distance between the two numbers; when the initials are different, the similarity between diseases i and j is 0.

3.3 Construction of Patient Portrait Similarity Network

The patient portrait similarity is calculated by the weighted average of the age similarity, gender similarity, ethnic similarity, allergen similarity, family history similarity, blood type similarity, historical diagnosis similarity, historical drug use similarity and abnormal test results similarity of patients. Generally speaking, it can be considered that the similarity weights of each dimension are the same. Among the above similarities, the age similarity is calculated by an Euclidean distance; the gender similarity and the ethnicity similarity are calculated using a binary approach where a value of 1 indicates similarity (when the gender or ethnicity is the same) and a value of 0 indicates dissimilarity. The information of other dimensions are calculated by using a Jaccard distance through coding.

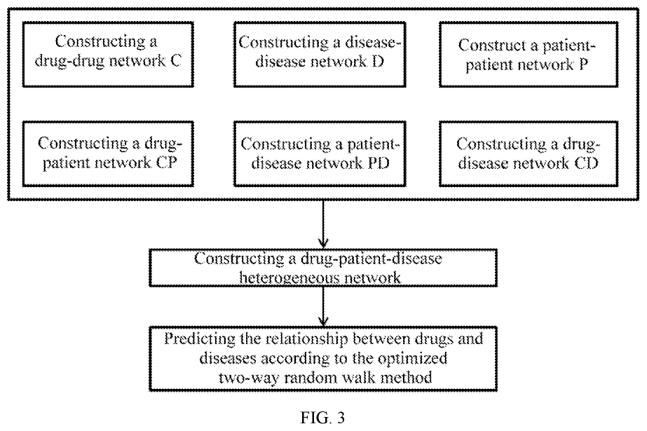

Step 4: discovery of new drug indications, as shown in FIG. 3, includes the following steps:

1) A drug-drug network C is constructed, with drug chemical components as network nodes and drug composite similarity as the edge of the network.

2) A disease-disease network D is constructed, with diseases as the nodes of the network and disease phenotype similarity as the edge of the network.

3) A patient-patient network P is constructed, with the patient portraits as the nodes of the network and the patient portrait similarity as the edge of the network; when the patient portrait similarity between two nodes is less than a threshold ε, the value of the edge between the two nodes is set to 0, and a quarter of the similarity of all patient portraits can be taken for ε.

4) A drug-patient relationship network CP is constructed, the patient medication data of the current visit after each patient portrait generated is extracted, and a drug-patient association dichotomy network B_{BP}(C, P, E) is constructed, where E(B_{cp})⊆P×C, E(B_{cp})={edges between e_{i,j}, cᵢ and pⱼ}, if a patient pⱼ uses a drug cᵢ in the current visit, the edge between cᵢ and pⱼ is set to 1, otherwise it is set to 0.

5) A patient-disease relationship network PD is constructed, the diagnosis data of the current visit after each patient portrait generated is extracted, and a patient-disease correlation dichotomy network B_{pd}(P, D, E) is constructed, where E(B_{pd})⊆D×P, E(B_{pd})={edges between e_{i,j}, pᵢ and dⱼ}; if the patient pᵢ is determined to have a disease dⱼ at the current visit, the edge between pᵢ and dⱼ is set to 1, otherwise it is set to 0.

6) A drug-disease relationship network CD is constructed, and a drug-disease association dichotomy network B cd (C, D, E) is constructed based on a SIDER data set, where E(B_{cd})⊆D×C, E(B_{cd})={edges between e_{i,j}, cᵢ and dⱼ}; if there is a known association between the drug cᵢ and disease dⱼ, the edge between the drug cᵢ and disease dⱼ is set to 1, otherwise it is set to 0.

7) Drug-patient-disease heterogeneous networks are constructed, including a drug-drug network, a disease-disease network, a patient-patient network, a drug-patient relationship network, a patient-disease relationship network and a drug-disease relationship network. The adjacency matrix A of the drug-patient-disease heterogeneous network can be expressed as:

$$A = [ \begin{matrix}{A}_{c} & {A}_{C P} & {A}_{C D} \\ {A}_{C P}^{T} & {A}_{P} & {A}_{P D} \\ {A}_{C D}^{T} & {A}_{P D}^{T} & {A}_{D}\end{matrix} ]$$

where A_{c}, A_{P} and A_{D} represent the adjacency matrices of the drug-drug network, patient-patient network and disease-disease network respectively, A_{CP}, A_{PD} and A_{CD} represent the adjacency matrices of the drug-patient relationship network, patient-disease relationship network and drug-disease relationship network respectively, and A_{CP}^{T}, A_{PD}^{T} and A_{CD}^{T} represent transpositions of A_{CP}, A_{PD} and A_{CD} respectively.

8) According to the optimized bidirectional random walk method, the relationship between drugs and diseases is predicted. Supposing that the drug-patient-disease heterogeneous network contains information of n drugs, x patients and m diseases, now, the new drug indications are predicted for the drug cᵢ, that is, the drug cᵢ and the disease dⱼ are predicted, j=1, 2, . . . , m that is, drug cᵢ is used as a seed of random walk to predict the probability R of reaching the diseases dⱼ when random walk reaches a steady state, and the dimension of R is n×m.

Firstly, the initial vector R⁽⁰⁾ at the starting time t=0 of the random walk, that is, the known relation between drugs and diseases, is constructed and the adjacency matrix A_{CD} with the drug-disease relationship network is normalized.

$${R}^{( 0 )} = {A}_{C D} = \frac{{A}_{C D}}{s u m ( {A}_{C D} )}$$

where sum(A_{CD}) is the sum of all elements in A_{CD}.

In the process of walking in heterogeneous networks, the random walk seed has a certain probability of moving to neighboring nodes in the current network, and also has a certain probability of walking to other networks. According to the present disclosure, the bidirectional random walk method is optimized in combination with clinical situations, and is extended and applied to the random walk problem of the drug-patient-disease heterogeneous network. Supposing two random walk links are performed:

a) Forward link: the seed starts from a certain node in the drug-drug network and walks through the patient-patient network to the disease-disease network, and after walking for a time t, the probability of the walk seed staying at each node is calculated as follows:

R_{F-CP}^{(t)}=(1−λ_{CP})A_{C}R_{F-CP}^{(t-1)}+λ_{CP}A_{CP}

R_{F-PD}^{(t)}=(1−λ_{PD})A_{P}R_{F-PD}^{(t-1)}+λ_{PD}A_{PD}

R_{F}^{(t)}=αR_{F-CP}^{(t)}R_{F-PD}^{(t)}+(1−α)A_{CD}

where a subscript F represents the forward link, λ_{CP} represents a probability of the seed transferring from the drug-drug network to the patient-patient network, λ_{PD} represents a probability of the seed transferring from the patient-patient network to the disease-disease network; R_{F-CP}^{(t)} and R_{F-CP}^{(t-1)} are probabilities that the random walk seed from the drug-drug network stays in the patient-patient network at times t and t−1, R_{F-PD}^{(t)} and R_{F-PD}^{(t-1)} are probabilities that the random walk seed from the patient-patient network stays in the disease-disease network at the times t and t−1 in the forward link. The last formula integrates the results of the above two random walks, and introduces a weight factor α to introduce the known drug-disease relationship into the random walk process to carry out overall regulation to prevent the random walk from being too lengthy. The value of the weight factor α is between (0,1).

b) Reverse link: The seed starts from a certain node of the disease-disease network, passes through the patient-patient network and walks to the drug-drug network, and after walking for a time t, the probability of the walk seed stays at each node is calculated as follows:

R_{B-DP}^{(t)}=(1−λ_{DP})A_{D}R_{B-DP}^{(t-1)}+λ_{DP}A_{PD}^{T}

R_{B-PC}^{(t)}=(1−λ_{PC})A_{P}R_{B-PC}^{(t-1)}+λ_{PC}A_{CP}^{T}

R_{B}^{(t)}=α(R_{B-DP}^{(t)}R_{B-PC}^{(t)})^{T}+(1−α)A_{CD}

where a subscript B represents the reverse link λ_{DP} represents a probability of the seed transferring from the disease-disease network to the patient-patient network, λ_{PC} represents a probability of the seed transferring from the patient-patient network to the drug-drug network; R_{B-DP}^{(t)} and R_{B-DP}^{(t-1)} are probabilities that the random walk seed from the disease-disease network stays in the patient-patient network at times t and t−1, R_{B-PC}^{(t)} and R_{B-PC}^{(t-1)} are probabilities that the random walk seed from the patient-patient network stays in the drug-drug network at the times t and t−1 in the reverse link. The function of the weighting factor is the same as that of the forward link.

In the network, assuming that nodes with more common neighbors are more closely related to each other and more likely to influence each other, a random walk length metric of nodes is constructed based on the topological structure of the heterogeneous network. On the one hand, it can make full use of different influences of different nodes on other contents in the heterogeneous network. On the other hand, it can help the random walk algorithm to converge quickly. The random walk length metric involved in the present disclosure is defined as follows.

In the forward link, the random walk lengths of the drug node cᵢ and patient node pᵢ are defined as L_{CP}(cᵢ) and L_{PD}(pᵢ); in the reverse link, the random walk lengths of the disease node dᵢ and the patient node pᵢ are defined as L_{DP}(dᵢ) and L_{PC}(pᵢ).

L_{CP}(cᵢ)=Σⱼ₌₁^{x}J(cᵢ,pⱼ)

L_{PD}(pᵢ)=Σⱼ₌₁^{m}J(pᵢ,dⱼ)

L_{DP}(dᵢ)=Σⱼ₌₁^{x}J(dᵢ,pⱼ)

L_{PC}(pᵢ)=Σⱼ₌₁ⁿJ(pᵢ,cⱼ)

Taking L_{CP} (cᵢ) as an example to explain the calculation method, J(cᵢ, pⱼ) is used to express the topological structure similarity of the nodes cᵢ and pⱼ, and is defined as follows:

$$J ( {c}_{i} , {p}_{j} ) = \frac{\vert {N}^{C} ( {c}_{i} ) ⋂ ( {p}_{j} ) \vert}{\vert {N}^{C} ( {c}_{i} ) ⋃ ( {p}_{j} ) \vert}$$

$$( {p}_{j} ) = {⋃}_{s ∈ ( {p}_{j} )} {N}^{C} ( s )$$

where N^{C}(cᵢ) represents the neighbor node of the node cᵢ in the drug-drug network C, and [symbol] (pⱼ) represents the neighbor nodes, in the drug-drug network C, of all the neighbor nodes of the node pⱼ in the patient-patient network P. In the iterative process of random walk, for cᵢ, at the time cᵢ, the random seeds starting from cᵢ will no longer walk. After the random walk, the final R is as follows:

$$R = \frac{( {R}_{F} + {R}_{B} )}{2}$$

That is, the probability that the drug can treat the corresponding disease; the greater the probability value, the greater the possibility that the drug can treat the disease in its corresponding (drug, disease) pair. If there is no known correlation between the two, the drug is considered to be the discovery result of the new indications of the drug. The hyperparameters α, A_{CP}, A_{PD}, A_{PC}, A_{DP} involved in the above calculation process can be obtained by cross-validation.

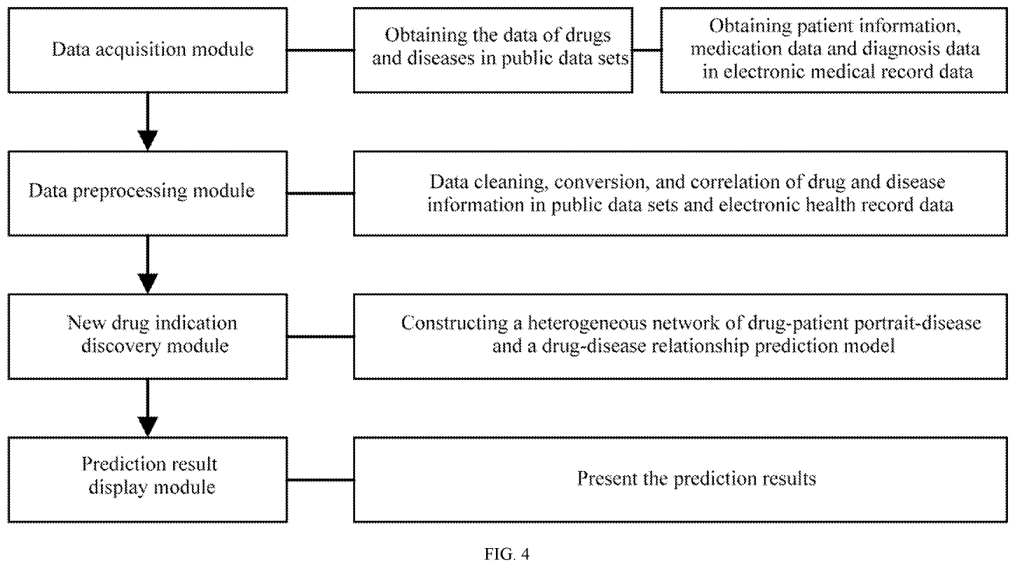

As shown in FIG. 4, an embodiment of the present disclosure provides a system for discovering new drug indications by fusing patient portrait information, which includes: a data acquisition module configured to acquire and associate public data of drugs and diseases and real-world patient data; a data preprocessing module configured to clean and convert data, and perform association mapping on the public data and the real-world patient data; a new drug indication discovery module configured to search for new drug indications in a drug-patient-disease global relationship; and a prediction result display module configured to present prediction result data. The new drug indication discovery module is the core module of the present disclosure. By using the method for discovering new drug indications, the performance of drugs and diseases in real-world clinical activities is correlated by constructing a patient portrait similarity network, and a heterogeneous network of drugs, patients and diseases is constructed, and then the relationship between drugs and diseases is predicted based on a bidirectional random walk method.

According to the present disclosure, real-world patient data is introduced, and the actual use and treatment of drugs in clinic are taken as important factors for drug repositioning prediction, so that the prediction result will be closer to the clinic, and the possibility of success in subsequent verification for new uses of old drugs and new clinical trials is greater.

In this application, the term “controller” and/or “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components (e.g., op amp circuit integrator as part of the heat flux data module) that provide the described functionality; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The above is only the preferred embodiment of the present disclosure, and although the present disclosure has been disclosed in the above with preferred embodiments, it is not intended to limit the present disclosure. Any person familiar with the field can make many possible changes and modifications to the technical solution of the present disclosure by using the methods and technical contents disclosed above, or modify it into equivalent embodiments with equivalent changes without departing from the scope of the technical solution of the present disclosure. Therefore, any simple modification, equivalent change and modification of the above embodiment according to the technical essence of the present disclosure that does not depart from the content of the technical solution of the present disclosure still falls within the scope of protection of the technical solution of the present disclosure.

What is claimed is:

1. A method for discovering new drug indications by fusing patient portrait information, comprising:
step (1) performing data acquisition and association: obtaining public data of drugs and diseases, obtaining real-world patient data from electronic health record data, and associating drugs and diseases in the real-world patient data with drugs and diseases in the public data corresponding to the real-world patient data;
step (2) generating patient portraits: generating corresponding patient labels by cleaning and converting the electronic health record data in step (1), wherein a same patient having a plurality of visits possesses a plurality of patient portraits;
step (3) calculating a drug composite similarity, a disease phenotype similarity and a patient portrait similarity, and constructing a drug-drug network C, a disease-disease network D and a patient-patient network P according to the drug composite similarity, the disease phenotype similarity and the patient portrait similarity;
step (4) constructing a drug-patient relationship network CP according to medication data of a current visit after each patient portrait generated, constructing a patient-disease relationship network PD according to diagnostic data of the current visit after each patient portrait generated, and constructing a drug-disease relationship network CD according to a known association between the drugs and the diseases;
step (5) constructing a drug-patient-disease heterogeneous network by the networks C, D, P, CP, PD and CD, wherein an adjacency matrix A of the heterogeneous network is:
$$A = [ \begin{matrix}{A}_{c} & {A}_{C P} & {A}_{C D} \\ {A}_{C P}^{T} & {A}_{P} & {A}_{P D} \\ {A}_{C D}^{T} & {A}_{P D}^{T} & {A}_{D}\end{matrix} ]$$
where A_{c}, A_{P} and A_{D} represent adjacency matrices of the networks C, P and D, respectively, A_{CP}, A_{PD} and A_{CD} represent adjacency matrices of the networks CP, PD and CD respectively, and T represents transposition; and
step (6) predicting a relationship between the drugs and the diseases based on a two-way random walk method, that is, taking a certain drug node as a seed of random walk, and predicting a probability R of reaching a certain disease node when the random walk reaches a steady state, comprising:
constructing an initial vector R⁽⁰⁾=A_{CD} at a random walk starting time t=0, and performing normalization on A_{CD};
assuming two random walk links, comprising:
a) a forward link: the seed starts from a certain node in the network C and walks through the network P to the network D, and after walking for time t, a probability of the walk seed staying at each node is calculated as follows:
R_{F-CP}^{(t)}=(1−λ_{CP})A_{C}R_{F-CP}^{(t-1)}+λ_{CP}A_{CP}
R_{F-PD}^{(t)}=(1−λ_{PD})A_{P}R_{F-PD}^{(t-1)}+λ_{PD}A_{PD}
R_{F}^{(t)}=αR_{F-CP}^{(t)}R_{F-PD}^{(t)}+(1−α)A_{CD}
where a subscript F represents the forward link, λ_{CP} represents a probability of the seed transferring from the network C to the network P, λ_{PD} represents a probability of the seed transferring from the network P to the network D, R_{F-CP}^{(t)} and R_{F-CP}^{(t-1)} represent probabilities that the random walk seed starting from the network C stays in the network P at the time t and time t−1, respectively, R_{F-PD}^{(t)} and R_{F-PD}^{(t-1)} represent probabilities that the random walk seed from the network P stays in the network D at the time t and the time t−1, respectively, and a represents a weight factor; and
b) a reverse link: the seed starts from a certain node in the network D and walks through the network P to the network C, and after walking for the time t, a probability of the walk seed staying at each node is calculated as follows:
R_{B-DP}^{(t)}=(1−λ_{DP})A_{D}R_{B-DP}^{(t-1)}+λ_{DP}A_{PD}^{T}
R_{B-PC}^{(t)}=(1−λ_{PC})A_{P}R_{B-PC}^{(t-1)}+λ_{PC}A_{CP}^{T}
R_{B}^{(t)}=α(R_{B-DP}^{(t)}R_{B-PC}^{(t)})^{T}+(1−α)A_{CD}
where a subscript B represents the reverse link, λ_{DP} represents a probability of the seed transferring from the network D to the network P, λ_{PC} represents a probability of the seed transferring from the network P to the network C, R_{B-DP}^{(t)} and R_{B-DP}^{(t-1)} represent probabilities that the random walk seed starting from the network D stays in the network P at the time t and the time t−1, respectively, and R_{B-PC}^{(t)} and R_{B-PC}^{(t-1)} represent probabilities that the random walk seed starting from the network P stays in the network C at the time t and the time t−1; and
calculating random walk lengths of a drug node and a patient node in the forward link, and random walk lengths of a disease node and a patient node in the reverse link, respectively, based on a topological structure of the heterogeneous network, wherein in random walk iteration, when a certain node satisfies that a random walk length of the certain node is smaller than or equal to t, the random seed starting from the node stops walking, and wherein
$$R = \frac{( {R}_{F} + {R}_{B} )}{2}$$
obtained when the random walk ends represents a probability of a drug treating a disease corresponding to the drug, and when the drug and the disease have no known association the drug is taken as a discovery result of new drug indications.

2. The method for discovering new drug indications by fusing the patient portrait information according to claim 1, wherein in step (1), the information obtained from the electronic health record data comprises:
demographic information: age, gender and ethnicity;
basic medical information: allergy history, family history and a blood type;
diagnosis and treatment information: historical diagnostic records, abnormal laboratory results and historical medication records; and
medical result information: diagnosis and medication records generated by the current visit.

3. The method for discovering new drug indications by fusing the patient portrait information according to claim 2, wherein in step (2), custom codes are applied to gender, ethnicity, allergen, blood type and abnormal test results of a patient, coding forms of the custom codes are not limited, ICD-10 codes are applied to historical diagnosis and family medical history, and drug codes in a DrugBank data set are applied to historical medication information.

4. The method for discovering new drug indications by fusing the patient portrait information according to claim 1, wherein in step (3), the drug composite similarity comprises a drug structure similarity, a target similarity, a pathway similarity and an adverse reaction similarity, a structural similarity of drugs is calculated by computing a Tanimoto coefficient using drug 2D molecular fingerprint data, and the target similarity, the pathway similarity and the adverse reaction similarity are obtained by calculating a Jaccard coefficient.

5. The method for discovering new drug indications by fusing the patient portrait information according to claim 4, wherein in step (3), said calculating the drug composite similarity comprises:
calculating the drug composite similarity using a nonlinear heterogeneous network fusion mode according to four dimensions of the drug composite similarity, wherein a similarity network of each dimension is expressed as G=(V, E), where V represents a node, corresponding to drugs in the four similarity networks, and E represents an edge, characterized by similarities among the drugs; and wherein an overall normalized weight matrix K for the four similarity networks is defined as follows:
$$K ( i , j ) = \{ \begin{matrix}\frac{s i m ( i , j )}{2 {\sum }_{k \neq i} s i m ( i , k )} , j \neq i \\ \frac{1}{2} , j = i\end{matrix}$$
where sim (i, j) is a similarity between a drug i and a drug j in a certain dimension;
defining a local weight matrix S as follows:
$$S ( i , j ) = \{ \begin{matrix}\frac{s i m ( i , j )}{{\sum }_{k ∈ {N}_{i}} s i m ( i , k )} , j ∈ {N}_{i} \\ 0 , other\end{matrix}$$
where Nᵢ represents a neighbor node of a node i calculated through a KNN algorithm, and a similarity among non-neighbor nodes is set as 0; and
wherein the calculated matrices K and S are taken as an initial state of the heterogeneous network fusion, and an iterative update formula for the heterogeneous network fusion for the similarity network of each dimension is as follows:
$${K}^{( v )} = {S}^{( v )} \times ( \frac{{\sum }_{k \neq v} {K}^{( v )}}{m - 1} ) \times {( {S}^{( v )} )}^{T} , v = 1 , 2 , … m , m = 4$$
obtaining a final drug composite similarity when K^{(v)} tends to be stable and consistent after a plurality of iterations.

6. The method for discovering new drug indications by fusing the patient portrait information according to claim 1, wherein in step (3), the disease phenotype similarity is calculated using a hierarchical coding structure of ICD-10, and wherein a disease phenotype similarity between diseases i and j is calculated as follows:
$$sim ( i , j ) = \{ \begin{matrix}1 - {‖ Number ( i ) - Number ( j ) ‖}^{2} & \begin{matrix}Initial letters of ICD - 10 codes \\ for i and j are the same\end{matrix} \\ 0 & other\end{matrix}$$
where Number(i) and Number(j) represent numbers after removing first letters from ICD-10 codes of the diseases i and j, respectively.

7. The method for discovering new drug indications by fusing the patient portrait information according to claim 1, wherein in step (3), the patient portrait similarity is calculated by weighted averaging of an age similarity, a gender similarity, an ethnic similarity, an allergen similarity, a family medical history similarity, a blood type similarity, a historical diagnostic similarity, a historical medication similarity and an abnormal testing result similarity of patients; the age similarity is calculated using a Euclidean distance; the gender similarity and the ethnicity similarity are calculated using a binary approach, wherein a value of 1 indicates similarity (when the gender or ethnicity of two patients are the same) and a value of 0 indicates dissimilarity, and information of other dimensions are calculated by using a Jaccard distance through coding.

8. The method for discovering new drug indications by fusing the patient portrait information according to claim 1, wherein in step (3), during constructing the patient-patient network P, when the patient portrait similarity between two nodes is smaller than a threshold value ε, a value of an edge between the two nodes is set as 0, wherein ε is set to be a quantile of all the patient portrait similarity.

9. The method for discovering new drug indications by fusing the patient portrait information according to claim 1, wherein in step (6), assuming that the drug-patient-disease heterogeneous network totally contains n drugs, x patients and m types of disease information, random walk lengths L_{CP}(cᵢ) and L_{Pd}(pᵢ) of a drug node cᵢ and a patient node pᵢ in the forward link, and random walk lengths L_{DP}(dᵢ) and L_{PC}(pᵢ) of a disease node dᵢ and a patient node pᵢ in the reverse link are calculated as follows:
$${L}_{C P} ( {c}_{i} ) = {\sum }_{j = 1}^{x} J ( {c}_{i} , {p}_{j} )$$
$${L}_{P D} ( {p}_{i} ) = {\sum }_{j = 1}^{m} J ( {p}_{i} , {d}_{j} )$$
$${L}_{D P} ( {d}_{i} ) = {\sum }_{j = 1}^{x} J ( {d}_{i} , {p}_{j} )$$
$${L}_{P C} ( {p}_{i} ) = {\sum }_{j = 1}^{n} J ( {p}_{i} , {c}_{j} )$$
where J represents a topological structure similarity of two nodes,
wherein J(cᵢ, pᵢ) for L_{CP}(cᵢ) is calculated as follows:
$$J ( {c}_{i} , {p}_{j} ) = \frac{\vert {N}^{C} ( {c}_{i} ) ⋂ ( {p}_{j} ) \vert}{\vert {N}^{C} ( {c}_{i} ) ⋃ ( {p}_{j} ) \vert}$$
$$( {p}_{j} ) = {⋃}_{s ∈ ( {p}_{j} )} {N}^{C} ( s )$$
where N^{C}(cᵢ) represents a neighbor node of the node cᵢ in the drug-drug network C, and [symbol] (pⱼ) represents a neighbor node of all neighbor nodes in the drug-drug network C of a node pⱼ in the patient-patient network P.

10. A system for discovering new drug indications by fusing the patient portrait information, comprising:
a data acquisition module configured to acquire and associate public data of drugs and diseases and real-world patient data;
a data preprocessing module configured to clean and convert data, and perform association mapping on the public data and the real-world patient data;
a new drug indication discovery module configured to search for new drug indications in a drug-patient-disease global relationship; and
a prediction result display module configured to present prediction result data,
wherein the new drug indication discovery module uses the method for discovering new drug indications according to claim 1 to construct a drug-patient-disease heterogeneous network, and predicts drug-disease relationship based on a two-way random walk method.
